(19) United States
(12) Patent Application Publication
(10) Pub. No.: US 2026/0198802 A1
(43) Pub. Date: Jul. 16, 2026

(54) CANINE ECG MONITORING VIA WEARABLE INERTIAL MEASUREMENT UNITS

(71) Applicant: North Carolina State University, Raleigh, NC (US)

(72) Inventors: David Roberts, Raleigh, NC (US); Jianxun Wang, Raleigh, NC (US); Alper Bozkurt, Raleigh, NC (US); Marc Foster, Raleigh, NC (US)

(21) Appl. No.: 19/135,537

(22) PCT Filed: Dec. 4, 2023
(86) PCT No.: PCT/US2023/082316

§ 371 Date: Jun. 4, 2025

Related U.S. Application Data
(60) Provisional application No. 63/430,040, filed on Dec. 4, 2022.

(51) Int. Cl. A61B 5/11; A61B 5/00

(52) U.S. Cl. CPC A61B 5/1102; A61B 5/7264; A61B 5/7278; A61B 2503/40

(57) ABSTRACT

Various examples are provided related to electrocardiogram (ECG) monitoring from inertial measurement unit (IMU) signals. In one example, a method includes obtaining seismocardiogram (SGD) signals from IMU attached to a canine, the SGD signals including a first subset of signals generated by the IMU prior to a target timestamp and a second subset of signals generated by the IMU after the target timestamp; extracting input features from the SGD signals; and generating reconstructed ECG signals from the extracted input features using at least one neural network, where the reconstructed ECG signals represent an ECG reading of the canine. In another example, a system includes one or more IMU that can provide SGD signals and processing circuitry that can receive SGD signals from the one or more IMU; extract input features from the SGD signals; and generate reconstructed ECG signals from the extracted input features.

### CROSS REFERENCE TO RELATED APPLICATIONS

This application claims priority to, and the benefit of, co-pending U.S. provisional application entitled “Canine ECG Monitoring Via Wearable Inertial Measurement Units” having Ser. No. 63/430,040, filed Dec. 4, 2022, which is hereby incorporated by reference in its entirety.

### BACKGROUND

Wearable electrocardiogram (ECG) sensors can detect dogs' heartbeat signals and have proven useful in monitoring dogs' welfare and predicting temperament. Despite advances in the ergonomics, performance, and usability of ECG sensor technologies specifically designed for dogs, deploying those systems in the real world imposes challenges such as training human operators to ensure electrodes' proper contact with the skin and, especially in the case of puppies, socialization to achieve comfort and reduce behavioral inhibition.

### SUMMARY

Aspects of the present disclosure are related to electrocardiogram (ECG) monitoring from inertial measurement units (IMU) signals. In one aspect, among others, a method for reconstructing canine electrocardiogram (ECG) signals from Inertial Measurement Unit (IMU) data comprises obtaining a plurality of seismocardiogram (SGD) signals from one or more IMU attached to a canine, the plurality of SGD signals comprising a first subset of signals generated by the one or more IMU prior to a target timestamp and a second subset of signals generated by the one or more IMU after the target timestamp; extracting a defined set of input features from each of the plurality of SGD signals; and generating reconstructed ECG signals from the defined set of extracted input features for each of the plurality of SGD signals using at least one neural network, each reconstructed ECG signal corresponding to a SGD signal of the plurality of SGD signals, wherein the reconstructed ECG signals represent an ECG reading of the canine. The method can further comprise rendering the reconstructed ECG signals for display on a device.

In one or more aspects, the at least one neural network can comprise an attention-based model. The attention-based model can comprise a self-attention mechanism comprising feature extraction layers configured to extract query, value and key components. The attention-based model can comprise a point-wise feed forward layer configured to generate the reconstructed ECG signals based upon an output of the self-attention mechanism. In various aspects, the at least one neural network can comprise a contrastive representation model. The contrastive representation model can comprise an embedding model configured to determine contrastive loss between data pairs and a downstream model configured to generate the reconstructed ECG signals based upon an output of the embedding model. The plurality of SGD signals can comprise three-axis accelerometer signals and three-axis gyroscope signals. The defined set of extracted input features can comprise normalized three-axis accelerometer signals and normalized three-axis gyroscope signals.

In another aspect, a system for reconstructing canine electrocardiogram (ECG) signals from Inertial Measurement Unit (IMU) data comprises one or more IMU configured for attachment to a canine, the one or more IMU configured to provide seismocardiogram (SGD) signals; and processing circuitry configured to: receive a plurality of SGD signals from the one or more IMU, the plurality of SGD signals comprising a first subset of signals generated by the one or more IMU prior to a target timestamp and a second subset of signals generated by the one or more IMU after the target timestamp; extract a defined set of input features from each of the plurality of SGD signals; and generate reconstructed ECG signals from the defined set of extracted input features for each of the plurality of SGD signals using at least one neural network, each reconstructed ECG signal corresponding to a SGD signal of the plurality of SGD signals, wherein the reconstructed ECG signals represent an ECG reading of the canine. The SGD signals can be wirelessly transmitted to the processing circuitry. The system can comprise a harness configured to secure the one or more IMU to the canine, the harness comprising sensor circuitry configured to transmit the SGD signals. The plurality of SGD signals can comprise three-axis accelerometer signals and three-axis gyroscope signals. The defined set of extracted input features can comprise normalized three-axis accelerometer signals and normalized three-axis gyroscope signals.

In one or more aspects, the at least one neural network comprises an attention-based model. The attention-based model can comprise a self-attention mechanism comprising feature extraction layers configured to extract query, value and key components. The attention-based model can comprise a point-wise feed forward layer configured to generate the reconstructed ECG signals based upon an output of the self-attention mechanism. In various aspects, the at least one neural network comprises a contrastive representation model. The contrastive representation model can comprise an embedding model configured to determine contrastive loss between data pairs and a downstream model configured to generate the reconstructed ECG signals based upon an output of the embedding model. The processing circuitry can be configured to render the reconstructed ECG signals for display on a device.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described embodiments are usable in all aspects of the disclosure taught herein. Furthermore, the individual features of the dependent claims, as well as all optional and preferred features and modifications of the described embodiments are combinable and interchangeable with one another.

### BRIEF DESCRIPTION OF THE DRAWINGS

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIG. 1A includes images illustrating an example of a data collection system on a puppy (top) and its components (bottom), in accordance with various embodiments of the present disclosure.

FIG. 1B illustrates another example of a data collection system on a collar (top) and its components (middle and bottom), in accordance with various embodiments of the present disclosure.

FIG. 2 illustrates an example of an ECG reconstruction pipeline, in accordance with various embodiments of the present disclosure.

FIG. 3 illustrates and example of a dot product self-attention mechanism, in accordance with various embodiments of the present disclosure.

FIG. 4 illustrates an example of a single building block of convolution-augmented attention-based model, in accordance with various embodiments of the present disclosure.

FIG. 5 illustrates an example of a model architecture for contrastive loss training, in accordance with various embodiments of the present disclosure.

FIG. 6 illustrates examples of ECG reconstruction results, in accordance with various embodiments of the present disclosure.

FIG. 7 illustrates examples of ECG reconstruction results with high IMU variance, in accordance with various embodiments of the present disclosure.

FIG. 8 illustrates examples of inertial measurement unit (IMU) acceleration change magnitude distribution across different sessions, in accordance with various embodiments of the present disclosure.

FIGS. 9A and 9B illustrate examples of windowed F1 score distribution over different sessions and relationship between F1 score and IMU acceleration magnitude change in windows, respectively, in accordance with various embodiments of the present disclosure.

FIG. 10 is a schematic diagram illustrating an example of processing circuitry that can be used for ECG reconstruction from IMU signals, in accordance with various embodiments of the present disclosure.

### DETAILED DESCRIPTION

Disclosed herein are various examples related to electrocardiogram (ECG) monitoring from inertial measurement units (IMU) signals. Seismocardiogram signal is an alternate modality for heartbeat signals and is acquired using the IMU, which is commercially available, widely deployed, and does not require skin-contact. However, the extracted signals from an IMU are subject to heavy influences from motion and other noise sources. A method is disclosed that extracts physiological parameters, similar to those provided by ECG, using easier-to-use IMU sensors and a machine learning framework that reconstructs ECG signals from signals obtained from the IMU even during moderate to heavy movements. The approach leverages artificial neural networks to overcome severe noise artifacts in the IMU signal resulting from dogs' movements and environmental factors. Reference will now be made in detail to the description of the embodiments as illustrated in the drawings, wherein like reference numbers indicate like parts throughout the several views.

Electrocardiogram (ECG) and inertial measurement are two common signals tracked by wearable canine physiological monitoring devices. They have received increasing interest from both the research and consumer product community. Through continuous real-time monitoring from a wearable device, ECG provides heart rate information, while inertial measurement units (IMU) present the raw or fused linear and angular acceleration data that can be used for inferring other significant information such as activity level and dog pose. Both signals can be adopted for applications such as canine health monitoring, dog emotion analysis and guide dog temperament analysis.

While both ECG and inertial measurements are signals with significant analytic values, ECG signals can be harder to acquire compared to IMU signals. Existing ECG sensors for dogs require multiple sensors or electrodes and additional ECG amplifiers to produce a good signal output, which increases the overall wearable device volume and power consumption. In addition, the equipping of canine wearable devices with ECG electrodes requires special handling to ensure the electrodes' firm contact with dog's skin despite the fur without causing discomfort to the dog. This need can limit the daily use of such devices by regular users.

Furthermore, puppies usually need to undergo an additional equipment socialization process to become mentally and physically accustomed to the additional weight and restraints posed by the wearable device and ECG electrodes. For example, at Guiding Eyes for the Blind (GEB), a large, nationally-prominent nonprofit guide dog school in the United States, the socialization process of equipping the wearable band with ECG sensors lasts for two weeks. During the process, the puppy starts by wearing an empty wearable band without any sensors, and staff members gradually add dummy weights that simulate the sensors. Such a process also provides the opportunity for the staff member to observe whether the puppy can proceed with future tests wearing the device without any discomfort. If the puppy shows consistent discomfort when wearing the dummy band, staff members will not be able to attach the wearable device to the puppy even after two weeks of the socialization process.

However, it is possible to extract heart rate or even ECG waveform peaks from IMUs placed on humans or dogs. An accelerometer in the IMU can be utilized to detect the seismocardiogram (SCG) signal, which is the measurement of micro-vibrations caused by the contraction of the heart and the ejection of blood into the vascular tree. A deep learning approach can be deployed to directly reconstruct the ECG signal from IMU signals for, e.g., sleeping and resting dogs. This implies that ECG sensors can be replaced with IMU to create a more lightweight wearable device for dog welfare monitoring. It can also reduce the burden for non-expert dog owners to use such a device. The potential of ECG reconstruction can also extend the usage of signals from heart rate monitoring to other applications such as heart rhythm disturbance detection.

The effective ECG reconstruction using IMU signals and completely replacing ECG sensors with IMUs on canine wearable monitor devices face two major challenges. The first one is that IMU signals and SCG contained within them are highly influenced by various interference, such as motion artifacts caused by the dog's movement or the sensor's relative movement and even other physiological signals like respiration. The effect of motion artifacts can increase significantly during scenarios involving dogs' heavy movements. Previous similar studies on humans have shown that signals from multiple IMU sensors positioned at different locations on the test subject can help remove motion artifacts. However, such an approach can also increase the volume of the wearable device and dogs' burden. The second challenge lies in the wide variety of exhibited signal patterns due to experimental conditions. Variations in different conditions of monitored dogs, such as different dog behaviors, various dog breeds, and differences in dogs' physiological status, can cause IMU to exhibit different signal patterns. Such a wide variety of patterns is an obstacle to building a generalized pipeline of ECG reconstruction with data-driven approaches such as machine learning techniques.

In this disclosure, deep learning techniques can be utilized to tackle the first challenge and reconstruct ECG from IMU for dogs under moderate to heavy movements. Dogs undergoing the guide dog temperament evaluation process at GEB are considered, and their ECG readings are reconstructed from IMUs when they perform various tasks during test sessions. Two deep learning model architectures were designed to alleviate the impact of motion artifacts and other noises in ECG reconstruction. The first one modified the multi-head self-attention model with convolution-1D layers. The second one adapted the contrastive representation learning into the regression tasks to learn a more robust embedding for the ECG reconstruction. Peak detection and alignment were performed to compare the ECG R-peaks in real and predicted ECG signals to compute F1 scores for different models. Overall, the attention-based model with convolutional components achieved an F1 score of 0.7181, and the contrastive representation learning model achieved an F1 score of 0.6112. In addition, a qualitative and quantitative analysis localized to different periods of data sessions further illustrated the influence of the motion noise on ECG prediction and the robustness of the proposed models in tackling such noise.

### Usage of IMU Signal

Since IMU signals from a wearable device capture the acceleration and rotation of the subject's body parts, it can be adopted for dogs' posture estimation, behavior recognition, emotion analysis, and evaluation analysis such as guide dog temperament evaluation. Posture estimation and behavior recognition can rely on IMU signals from a single IMU sensor or multiple sensors attached to different locations on the dog. The received IMU signals can be analyzed using supervised learning algorithms such as, e.g., Random Forest, K-Nearest Neighbors, Support Vector Machine, Naïve Bayes, and Logistic Regression Models, to categorize the dog's state into one of the pre-defined postures or behaviors. Clustering algorithms can also be used to group IMU signals in an attempt to discover undefined postures. Based on the strong correlation between dogs' tail movement and their emotion status, the behavior recognition may be extended to emotion analysis by analyzing IMU signals from a sensor placed on the dog's tail. Because IMU signals can reflect the dog's status during different evaluation tasks and response towards different stimuli, a Neural Network model using IMU signals may be deployed as input to predict the suitability of a puppy being a future guide dog.

Due to the low-cost and low-power-consumption properties of IMU sensors, the IMU can also be utilized to monitor various vital signs. For example, the Savitzky-Golay filter and peak detection algorithms may be applied to IMU signals to estimate the respiration rate of dogs and humans, respectively. It has also been demonstrated that IMU signals can be used to directly reconstruct respiration signals through a convolutional neural network. In addition, advances in seismocardiography suggest that IMU sensors can be used to detect seismocardiogram signals, which reflects the subject's heart rate information. The use of IMU signals to estimate heart rate will be discussed in the next section.

### ECG and Heart Rate Analysis

Common signal sources of the heart rate analysis include ECG, photoplethysmogram (PPG), and seismocardiogram (SGD). ECG is usually considered the ground truth of the heart rate signals. However, its recording can be difficult in dog welfare monitoring due to the additional electrode-body contact requirement and a more complex device equipment process. PPG is a volumetric optical measurement that detects heartbeat from changes in the reflected infrared light from the blood flow. Good contact is needed between the optical elements and skin which is affected by the motion and presence of fur. Wavelet transformation and signal filtering can be utilized to remove the noise from PPG signals and the peak detection algorithm can be applied for final heart rate estimation on, e.g., pigs and dogs.

In this disclosure, ECG modeling uses SGD signals produced by IMU sensors. Previously, the heart rate for resting dogs has been monitored through IMU. The built-in IMU sensor of a smartphone was utilized to collect SGD signals and estimated the dog's heart rate when it was still and not panting through the peak detection algorithm and autocorrelation methods. IMU signals could also be collected through the sensor on a custom-designed dog collar system and bandpass filters applied before conducting heart rate estimation. ECG signals may also be reconstructed from IMU for resting dogs by applying deep learning models such as a convolutional neural network.

Heart rate analysis and ECG reconstruction using IMU signals for moving dogs impose a more demanding challenge because of the interference from various sources such as motion artifacts. Similar studies on humans suggest that multiple IMU sensors placed at different locations on the test subject can help alleviate this problem. Combining signals from two IMU sensors through the analog and digital processing, such as addition, subtraction, Euclidean norm, and/or absolute difference from the z-axis, can be used to amplify heartbeat information or suppress motion noise. Constrained independent component analysis can be utilized to decompose the signals from two IMU sensors into cardio-related signals and movement-related signals (noise). Here, a single IMU placement approach is described to reconstruct ECG signals from IMU using deep learning methods.

### Data Collection

Staff members at GEB collected data using a specialized system during puppies' temperament evaluation test sessions. In these tests, puppies with ages approximately 7.5 weeks-old went through a fixed-order set of tasks designed to examine puppies' reactions to various stimuli. Each evaluation session lasted around 15 minutes, and the dataset consisted of 133 recorded sessions. Examples of evaluation tasks include the dog walking up and down stairs and a can dropping behind the dog as it moves away to see the dog's response.

During the test session, the dog may perform various behaviors depending on the task and the handler's action. Typical dog behaviors annotated by staff members include sitting, standing, walking, trotting, running, chasing, scratching, sniffing, etc. The wide variety of dogs' behavior and corresponding motion magnitude will reflect in the recorded IMU signals and increase the difficulty of predicting ECG signals.

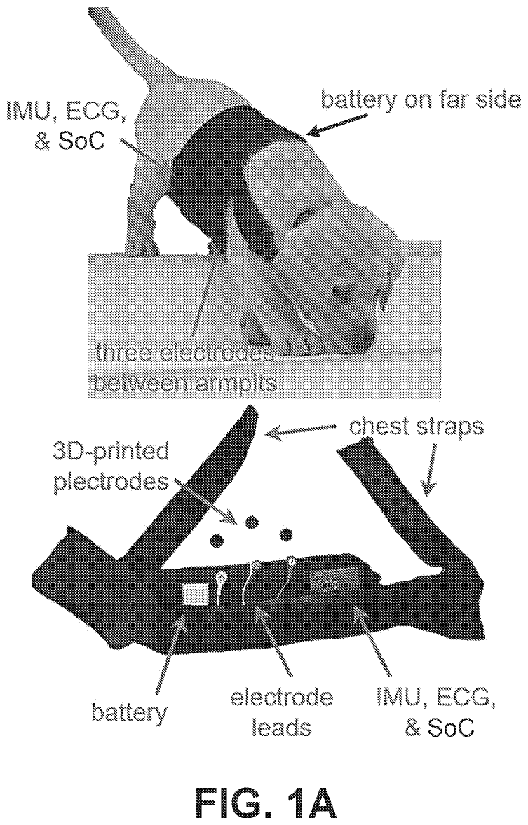

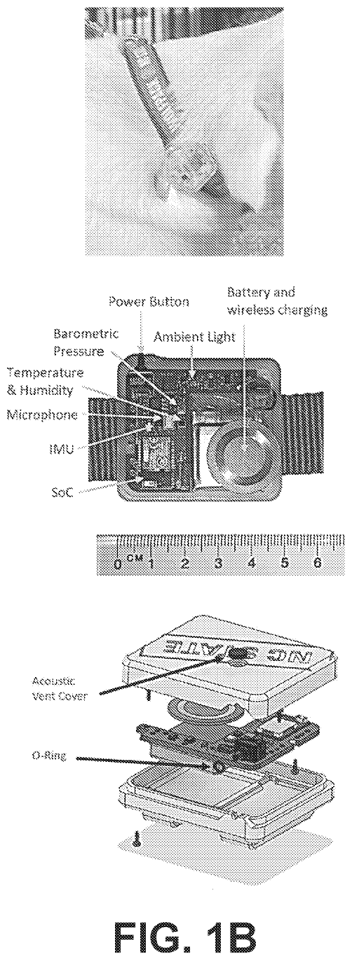

FIG. 1A illustrates an example of a data collection system on a puppy (top) and its components (bottom). The data collection system utilizes an infrastructure including on-body and off-body components. Here, relevant components are shown for ECG and IMU data collections. The on-body component comprises a strap placed around the dog's chest, which contains a microprocessor, sensors, electrodes and a battery. The ECG can be acquired through an integrated circuit front end (AD8232, Analog Device) and 3D printed custom electrodes. The analog signal from the ECG sensor can be digitized by an analog-to-digital converter chip (ADS1015, Texas Instruments). The IMU (LSM9DSO, STMicroelectronics) detects three-axis linear acceleration and three-axis angular acceleration signals. Both ECG and IMU circuits stream signals to the microprocessor for transmission. The microprocessor is a Raspberry Pi Zero Wireless with a 1 GHz Broadcom BCM2835 processor, a 512 MB LPDDR2 SDRAM, and an 8 GB SD card storage. It transmits the signal to a remote data aggregator in real-time through built-in wireless IEEE 802.11N transceiver. The data aggregator is a Dell laptop workstation with a custom-built application on the Universal Windows Platform developed in C#. It receives and records acquired data such as ECG and IMU signals, captured videos, and behavior annotations.

FIG. 1B illustrates another example of a data collection system on a collar (top) and its components (middle and bottom). As in the previous data collection system, this collar data collection system can utilize an infrastructure including on-body and off-body components. Here, components are shown for IMU data collections. The on-body component comprises a collar placed around the dog's neck, which contains a microprocessor, sensors, electrodes and a battery. The IMU (LSM9DSO, STMicroelectronics) can detect three-axis linear acceleration and three-axis angular acceleration signals. In addition, the data collection system can be configured to collect GPS location, activity level, barometric pressure, ambient light, ambient noise, ambient temperature, relative humidity levels and/or barking behavior based upon acoustic monitoring. The IMU circuit can stream signals to the microprocessor for transmission. The microprocessor can be a Raspberry Pi Zero Wireless with a 1 GHz Broadcom BCM2835 processor, a 512 MB LPDDR2 SDRAM, and an 8 GB SD card storage. It can transmit the signal to a remote data aggregator in real-time through built-in wireless IEEE 802.11N transceiver. The data aggregator can be a Dell laptop workstation with a custom-built application on the Universal Windows Platform developed in C#.

Ethics. The Institutional Animal Care and Use Committee (IACUC) reviewed and approved the data collection process and system. Collaborating with GEB staff and members of the Veterinary Behavior Service, a process was designed for socializing the puppies to the data collection system, as well as guidelines for staff to either terminate the socialization or end data collection experiments early. The protocol included several safety mechanisms to protect the dogs. Around three-weeks before the temperament evaluation test, the puppies were habituated to wearing the data collection system. The process started with an empty strap and progressively added additional weights and dummy sensors such that the puppies learned to tolerate the system and have a chance to show any discomfort that would prevent them from continuously using the wearable system. If the staff discovered that a puppy could not adapt to the system, the puppy participated in the test without using the system, and the record was not included in this study. During the test, if the staff noticed signs of distress or behavioral inhibition, the staff would remove the system from the dog and record only other off-body data sources, such as video and behavior annotations.

### Data Processing

Collected data take the form of multivariate time sequences with analog timestamps. The sampling frequency for ECG signals can range from about 225 Hz to 260 Hz, and the sampling frequency for IMU signals can range from about 120 Hz to 138 Hz. ECG and IMU signals were re-sampled and synchronized into 120 Hz frequency with digitized timestamps to ensure adjacent data points have the same elapsed time. Missing data was interpolated using PCHIP 1-D monotonic cubic interpolation for each axis, respectively.

The IMU data contains both three axes of accelerometer and three axes of gyroscope signals. To standardize and extract the signal changes in each axis respectively, each axis of IMU signals can be normalized over 200 timestamp windows, which corresponds to around 1.6 s. Dogs were frequently moving during test sessions, and the orientations for IMU signals could change in the middle of a data session. Therefore, six additional features were also included by normalizing among three accelerometer signals and three gyroscope signals respectively at each timestamp to provide orientation information and maintain relative magnitude among different axes. This resulted in a total of 12 features extracted from IMU at each timestamp. For ECG data, a similar windowed normalization was performed over 1000 timestamp windows, which is around 8.3 s, to alleviate the numeric impact of R-peaks in ECG and acquire a more stable signal.

### ECG Reconstruction Architecture

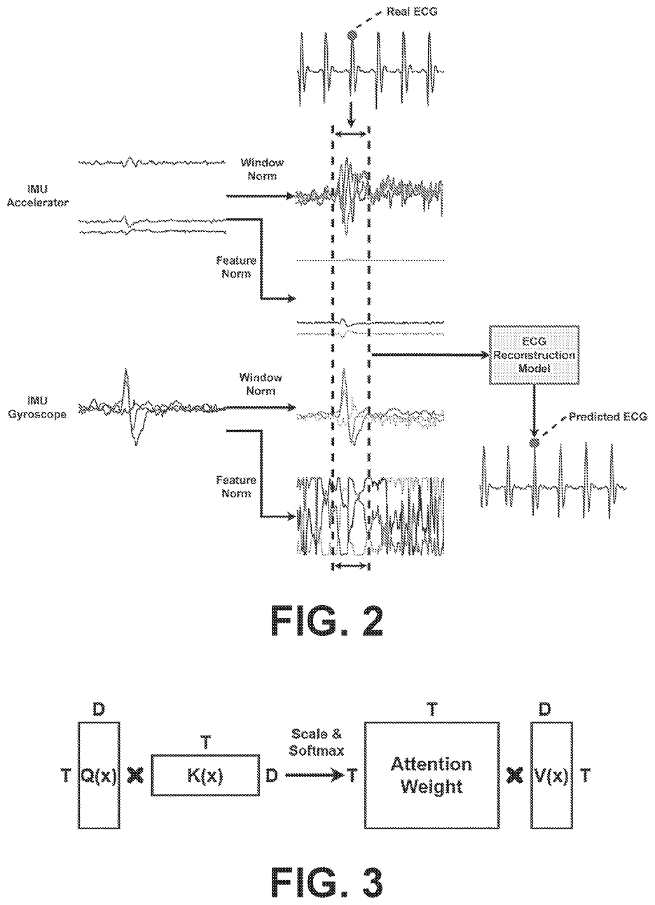

The ECG reconstruction problem can be formulated as a regression task and deep learning models applied to directly predict continuous ECG signal value at each timestamp given the input features extracted from IMU signals. Instead of modeling the ECG and IMU signal sequences, the sequence can be broken down and the ECG considered for one timestamp at a time. At each timestamp, the direct input for the model can be a windowed segment of processed IMU signals from 25 time steps before and 25 steps after the target timestamp. The input data take the form of a 50×12 matrix with 50 being the temporal dimension size and 12 being the feature dimension size. FIG. 2 illustrates an example of the high-level pipeline for predicting the ECG value at the timestamp indicated by the red dot (“Predicted ECG”). It is expected that such formulation can include sufficient temporal information without further interference from further away IMU signals. Models can be updated using stochastic gradient descent to minimize the Mean Square Error (MSE) between the model output and the target ECG signals.

Several model architectures were experimented with to reconstruct ECG signals, and two of them showed better performance compared to the baseline. The baseline architecture is based on the 1D convolution layers with L2 regularization. It contains two 1D convolution layers with 200 kernels and the kernel size as 5, followed by a flattening operation and two dense layers, each with 250 neurons. Each layer uses rectified linear unit (ReLU) as the activation function and is followed by a batch normalization layer.

### Attention-Based Model

The first architecture is based on the self-attention mechanism, which is a re-weighting method for sequence data. The self-attention mechanism uses a scoring function to compute the compatibility between data points at different locations in the sequence. It then uses the computed scores to scale the data points at each time step. The scoring function in this study is the dot product function. FIG. 3 illustrates an example of a dot product self-attention mechanism.

Consider a window of IMU data x as a T×D₀ matrix, where T is the size of the time window and D₀ is the initial signal dimension. In each block of attention mechanism, the input data x is encoded as three distinct matrices with Q(x) as query, K(x) as key, and V(x) as value through three separated feature extraction layers to expand the signal dimension to D. The matrix production Q(x)×K(x)^{T} produces the attention weight matrix representing the compatibility between each pair of timestamps. The final output of the self-attention mechanism is a T×D matrix that results from the matrix multiplication between the attention matrix and V(x). In the experiment, T=50, D₀=12 and D=250. The attention weight was scaled and normalized through softmax by the row dimension of the matrix to avoid changing the sum of values across the temporal dimension.

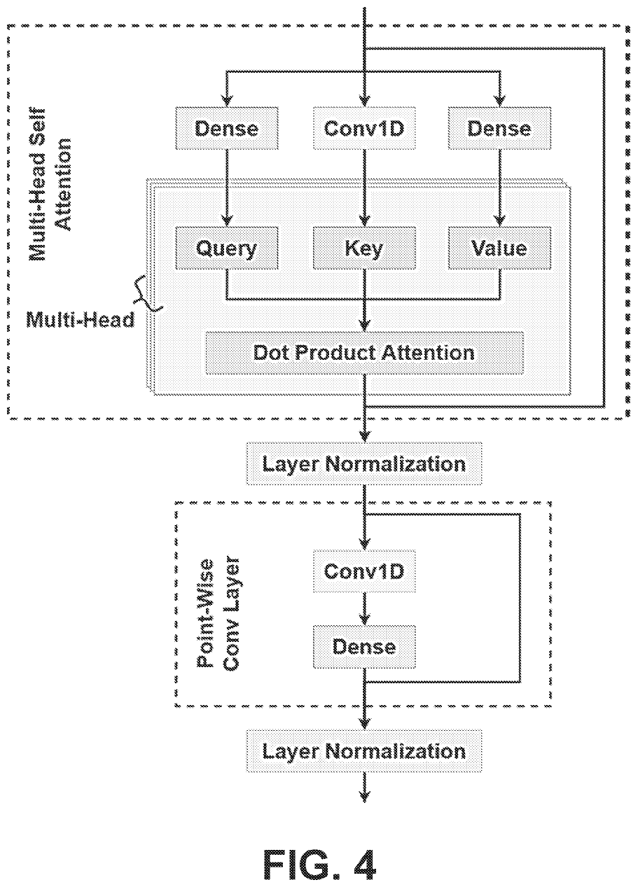

Two variants of the attention model were experimented with. The base version contained an encoding layer, two blocks of components illustrated in FIG. 4 except all 1D convolutional layers (Conv1D) are replaced with feedforward layers (Dense), and a final output layer. The encoding layer comprises a trainable Dense layer and a trigonometric position encoding added to the output of the trainable layer. The purpose of the trigonometric positional encoding is to maintain the positional information after the self-attention mechanism computation. In the base version, the feature extraction layers for query, value, and key components are all Dense layers. The multi-head architecture was adopted to compute multiple attention metrics at a time to separate the feature learning into several branches and set the number of heads to be 10. The multi-head self-attention mechanism is followed by a layer normalization. The model passes the output to a point-wise feedforward layer that comprises two Dense layers with 250 neurons applied to each time step followed by a layer normalization. Such a complex and deep model structure can cause gradients to be close to zero in some components of the model, which is a problem known as the vanishing gradient problem. To avoid this, a residual operation was included for both the attention component and the point-wise feedforward layer, which directly adds the skipped input values to the component's output. In addition, a 20% dropout layer was added after both the attention component and the point-wise feedforward layer to reduce the effect of overfitting. All layers used the ReLU activation function.

The second variant of the attention model is a combination of the convolutional neural network and the attention-based model which replaced the Dense feature extraction layer for the value component with a convolutional layer. In the second variant, three layers in the base variant are replaced with Conv1D layers: the input encoding layer, the feature extraction layer for the key component, and the first layer in the point-wise forward layer (see FIG. 4). All Conv1D layers have a kernel size of 5 and use padding to ensure the sequence length stays the same throughout the computation. The number of kernels for the Conv1D input encoding layer and point-wise feed forward layers is 250. The Conv1D variant of the attention model can be considered as an extension of the base variant since a Dense layer is equivalent to a Conv1D layer with a kernel size of 1 when applied to temporal sequences. The introduction of the convolution operation to the attention mechanism can increase the model's capability in capturing important information in IMU that corresponds to ECG signals.

For both variants of the attention model, the model was trained for 100 epochs with a batch size of 256. The Adam optimizer was used with the learning rate as 1e-4 to perform the training.

### Contrastive Representation Learning

The second architecture is based on contrastive representation learning. Contrastive representation learning is a self-supervised learning framework for the classification task that learns embeddings from input data by decreasing the distance measure between embeddings belonging to the same class (positive pairs) and increasing the distance measure for those belonging to different classes (negative pairs). Cosine distance, which is defined as one minus cosine similarity, was selected to be the distance measure. There are a wide range of loss functions for contrastive representation learning to achieve the aforementioned distance property among embeddings. Contrastive loss has the form:

$${ℒ}_{contrastive} = \frac{1}{{N}_{p}} \sum {[ {d}_{p} - {m}_{p} ]}_{+} + \frac{1}{{N}_{n}} \sum {[ {m}_{n} - {d}_{n} ]}_{+} , \qquad ( 1 )$$

where dₚ indicates distances between positive pairs and dₙ indicates distances between negative pairs. mₚ and mₙ represent the positive and the negative margin, respectively. Nₚ and Nₙ denote the total number of positive and negative pairs in the batch. In this study, mₚ=0 and mₙ=1. The subscription of + for the first and the second term indicates that only positive values are included in the summation and loss computation. The objective of the contrastive loss is to minimize the distance between positive pairs and increase the distance between negative pairs until the distance is larger than 1.

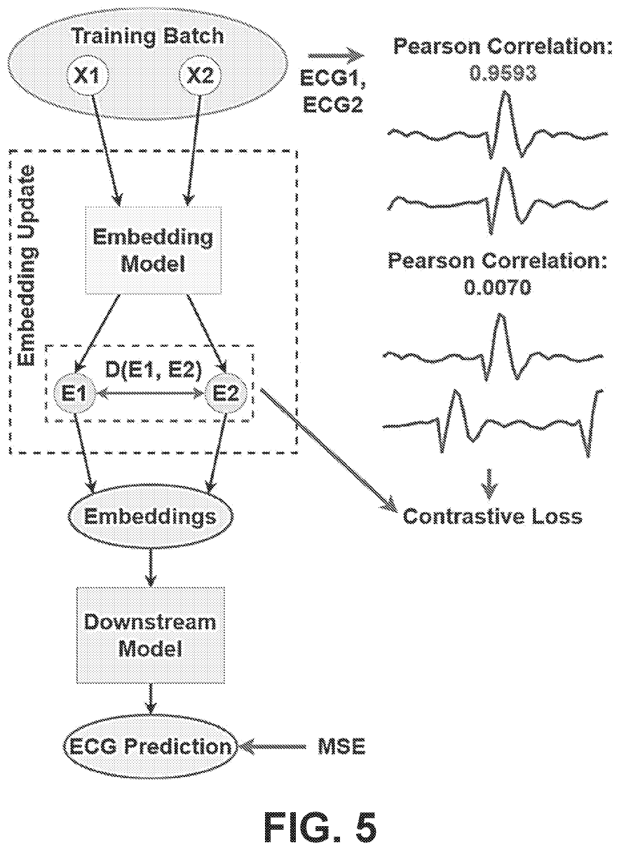

Because contrastive representation learning was originally designed for the classification problem, the following procedure can be applied to introduce the concept of positive/negative pairs to the ECG reconstruction problem and adapt it to contrastive representation learning. In addition to the windowed IMU segment data and ECG value for each timestamp, the ECG segment was extracted with the same window as IMU input data. During the loss calculation, the algorithm performs a pattern matching for each pair of data instances by computing the Pearson correlation between their ECG segments. If the Pearson correlation is greater than 0.8, such a pair is considered a positive pair; otherwise, it is considered a negative pair. FIG. 5 illustrates examples of a positive pair and a negative pair on the right. To speed up the training process and save the data storage space, we adopt an online pair generation for the positive and negative pairs used in the loss function. Instead of computing all pair labels from the entire dataset before the training process, the generation of positive and negative pairs only uses data instances within each batch during the training process.

The deployed architecture of the contrastive representation learning comprises an embedding model and a downstream model, as shown in FIG. 5. The embedding model follows the contrastive representation learning framework. It contains two 1D convolution layers with 200 kernels and the kernel size as 5, followed by a flattening operation. Each Conv1D layer in the embedding model uses ReLU as the activation function and is followed by a batch normalization layer. The output of the embedding model is a 240-size vector that is the computed embedding. The downstream model comprises two Dense layers, each with 120 neurons and the ReLU activation function. The structure of the second architecture is similar to the baseline 1D convolutional architecture but without the L2 regularization.

For a given timestamp and corresponding IMU input features Xi, the embedding model first produces the corresponding embedding Et, and then the downstream model makes the final ECG value prediction using E; as input. The training process backpropogates the weight update for the embedding model using the contrastive loss with the IMU segment as input and the weight update for the downstream model using the MSE loss with the computed embedding as input. The gradient calculated from the MSE regression loss is not applied to the embedding model. The model was trained for 100 epochs with a batch size of 1024 since positive pairs are easier to find with a larger batch size. The optimizers for both the embedding and downstream models are Adam optimizers. The learning rate for the embedding model starts at 3e-4 and linearly decrease to 1e-4 at step 1e5, which is around 53 epochs. The learning rate for the downstream model is 1e-4.

### Experimental Results

To train and evaluate the proposed ECG reconstruction models, 10 data sessions were selected from the entire dataset collected during the guide dog temperament test conducted at GEB. Because the data collection contained puppy activities at various levels, the collected ECG can comprise a considerable amount of noise. To ensure the quality of ECG as target signals for deep learning model training, the session selection was performed by the following procedures. Each data session in the GEB data was partitioned into 1.5 s sliding windows with a 0.5 s stride. Peak detection was applied with a distance threshold at 0.05 times the sampling rate, leading to 1200 beats per minute (bpm). Such loose constraint could lead to an unreasonably large number of peaks detected for noisy windows. Both the number of peaks and the R-R intervals were checked in each window to mark poor quality windows. In a signal window with good ECG quality, the number of peaks and R-R intervals were required to satisfy the condition of a minimum heart rate of 60 bpm and a maximum heart rate of 250 bpm. Ten data sessions were selected that contain over 90% good windows.

A 10-fold cross-validation was conducted to evaluate and compare different proposed ECG reconstruction models. The entire dataset was split into ten folds by shuffling all timestamps across all sessions and assigning each timestamp into one of the subsets. Each timestamp corresponds to the ECG value for that timestamp, the windowed IMU segment, and the windowed ECG segment for the contrastive representation learning model. For each subset, a standalone model was trained using the rest of the data and used to predict ECG values for this subset. Predicted ECG values for subsets were matched with original timestamps and formed the ECG prediction sequence for each session.

In the following sections, Conv1D, ATT, Att-Conv1D, and Contrast-Conv1D are used to indicate the baseline convolution 1-d model, the base attention-based model, the convolutional-layer-augmented attention-based model, and the convolutional model trained using contrastive representation learning, respectively.

### Qualitative Analysis

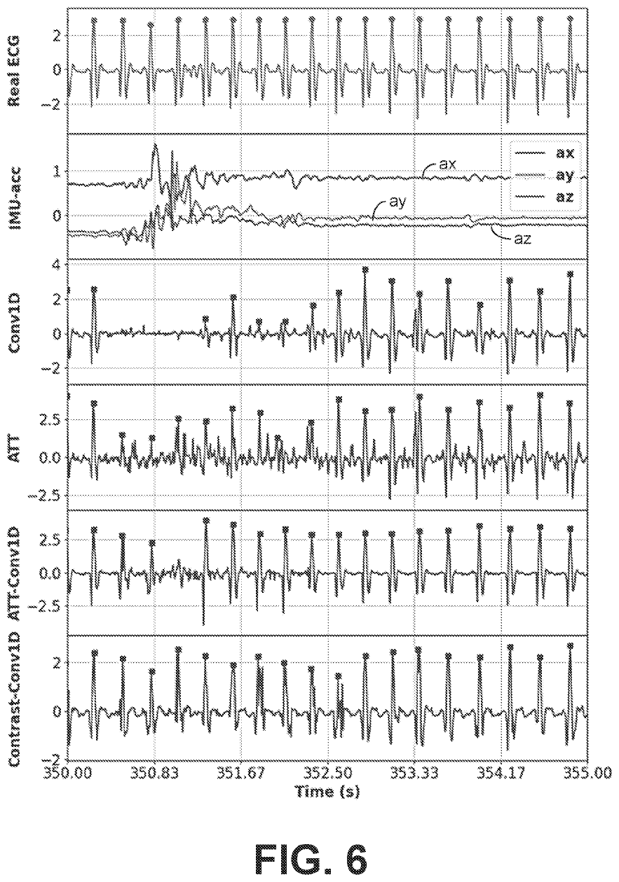

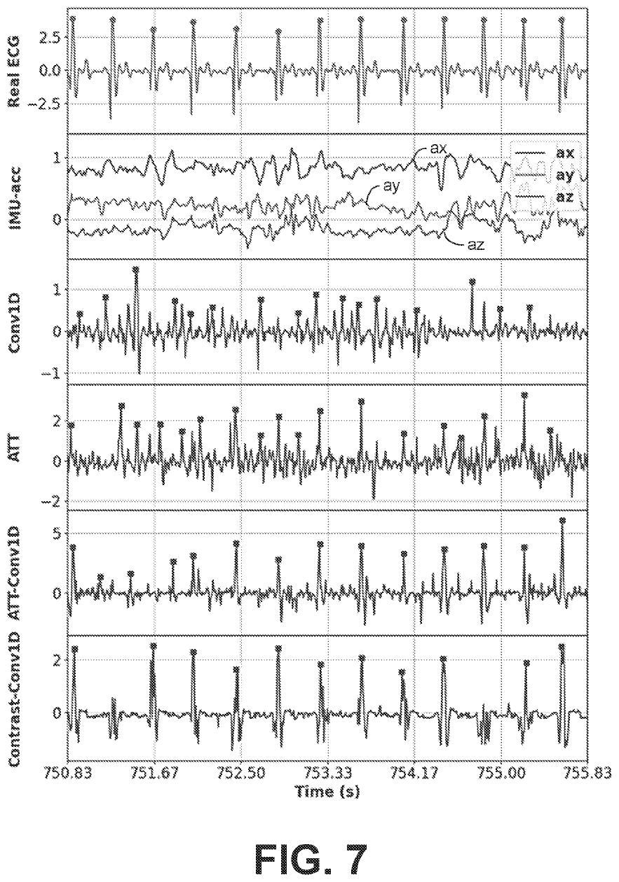

In FIGS. 6 and 7, the effect of ECG prediction from different models are first illustrated by comparing the QRS complexes, which is an ECG wave pattern related to a heartbeat, in the real ECG stream and predicted ECG signal streams. In a QRS complex, the R-peak has the highest value in the middle, which indicates a heartbeat. The Q-wave and S-wave are the signal drop before and after the R-peak, respectively. Several time periods were selected in the sessions with different motion levels to compare different models' output and inspect the effect of the motion level on the quality of models' prediction. The figures include three axes of IMU acceleration signals to capture the magnitude of dog motion. Along each ECG signal stream, R-peaks are marked by dots to show the result of applying peak detection with 0.15 times the sampling rate as the distance threshold, corresponding to 400 bpm. This shows the result of the R-peak detection algorithm with normal parameters and reflects noises in detected peaks.

FIG. 6 shows an example of ECG prediction from different models. All four models can produce visible QRS complexes after 352.5 s, where the corresponding IMU accelerometer signals are relatively stable. Comparing ECG signals produced by different models, signals from the Conv1D model and the ATT-Conv1D model are the least noisy, though R-peaks produced by the Conv1D model have a less consistent magnitude than those produced by the ATT-Conv1D model. The Contrast-Conv1D model can predict stable ECG signals in regions other than QRS complexes, but its QRS complexes are noisier compared to other models' produced signals. The ECG from the ATT model is the noisiest overall.

FIG. 7 illustrates a period with more significant IMU changes, compared to FIG. 6. Neither Conv1D nor ATT model can produce reasonable QRS complexes. Their output appears to be extremely noisy, with many false spikes that do not correspond to R-peaks. On the other hand, output signals from both ATT-Conv1D and Contrast-Conv1D models still relatively resemble the real ECG signals. In comparison, ATT-Conv1D's prediction is noisier, and its R-peak values are much higher than the real normalized ECG R-peak values. Predicted ECG from Contrast-Conv1D still contains noise in QRS complexes and misses some R-peaks, such as at around 751 s and 754.9 s. However, the corresponding prediction shows downward deflections that could correspond to Q and S waves, which indicates the embeddings could still identify the QRS complex to some extent. The ECG predictions in FIG. 7 suggest that ATTConv1D and Contrast-Conv1D are resilient to the influence of heavy motions but are still affected.

### Quantitative Analysis

To quantify the result and illustrate the effect of motion artifacts, a sliding window was applied with a 200 size and a 100 stride on every data session to generate segments of signals. Such segmentation allows a distribution of statistics to be generated from each window and the potential relationship examined between different statistics. The IMU acceleration change magnitude was used to indicate the severity of motion artifact, computed by the following equation:

$$\vert Δ {IMU}_{acc}^{i} \vert = \sqrt{{( {a}_{x}^{i} - {a}_{x}^{i - 1} )}^{2} + {( {a}_{y}^{i} - {a}_{y}^{i - 1} )}^{2} + {( {a}_{z}^{i} - {a}_{z}^{i - 1} )}^{2}} , \qquad ( 2 )$$

where

$${a}_{x}^{i} , {a}_{y}^{i} and {a}_{z}^{i}$$

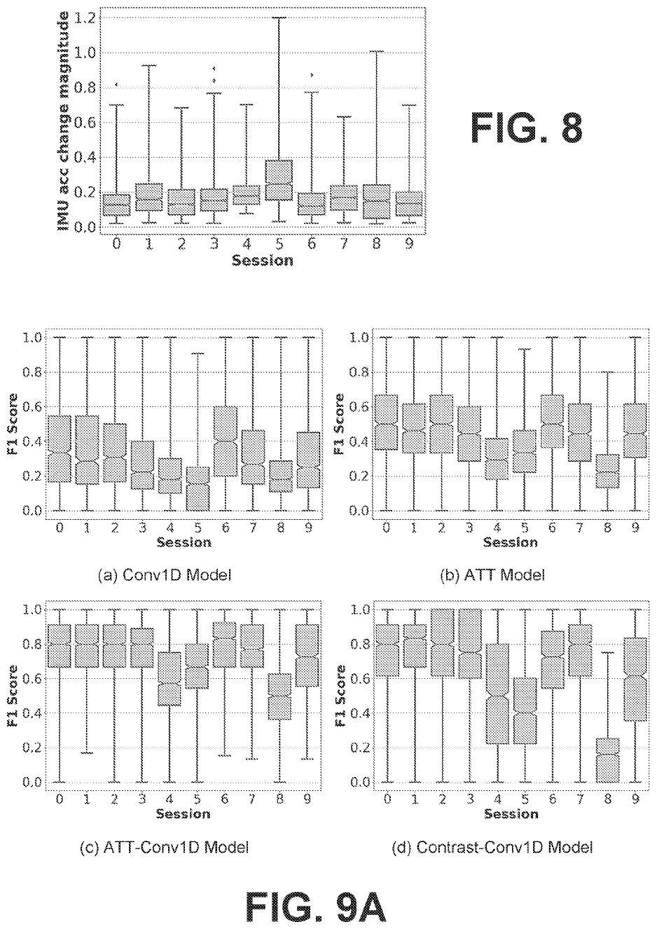

indicate the IMU acceleration value along x, y and z axis at timestamp i. The average IMU acceleration change magnitude was calculated in each window and its distribution plotted across different sessions in the format of a boxplot in FIG. 8. Out of all sessions, session 5 is the most unstable one since it has the highest median value (the middle line of the box) and the top 5-percent quantile (top whiskers). Session 4's overall IMU change magnitude distribution is more concentrated toward high values.

To quantify the models' performance, the R-peak detection and alignment was used with a stricter requirement to ensure the predicted ECG values are in the correct range. The parameters for the peak alignment algorithms are 0.05 times the sampling rate as the distance threshold and one error window tolerance. It is stricter than previous studies since the ECG prediction for each session came from different models, and such a setting can better identify noise in the prediction. The heart rate estimation accuracy was excluded from performance measurements for the same reason. By identifying a peak alignment as a true positive (TP), miss-aligned true R-peaks as false negatives (FN), and misaligned predicted R-peaks as false positives (FP), it was possible to compute precision, recall and F1 scores of deployed models using the following equations. The Mean Squared Error (MSE) between predicted and real ECG values in the overall result were also examined.

$$precision = \frac{TP}{TP + FP} \qquad ( 3 )$$

$$recall = \frac{TP}{TP + FN} \qquad ( 4 )$$

$$F 1 = \frac{2 * precision * recall}{precision + recall} \qquad ( 5 )$$

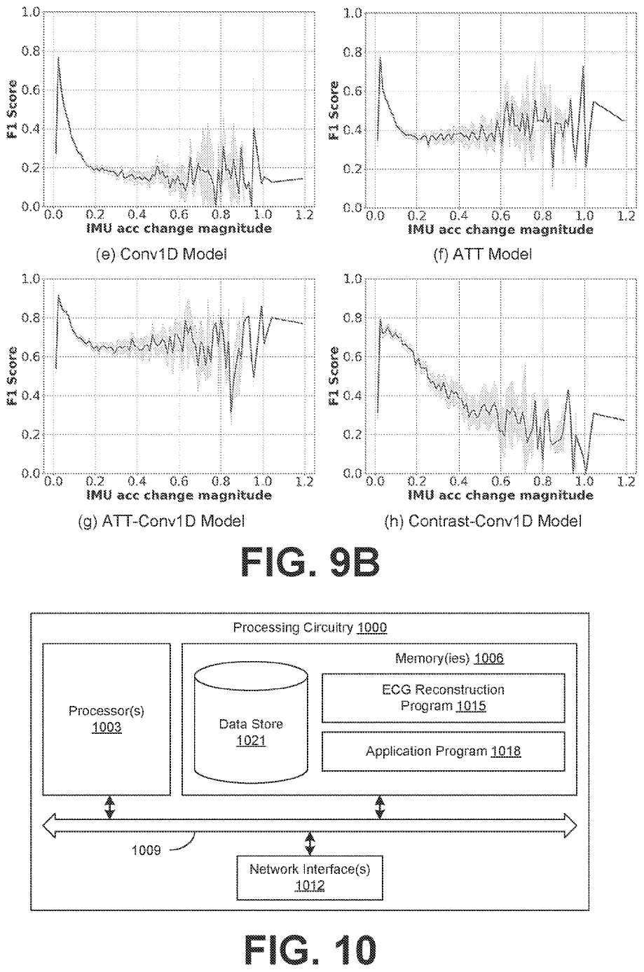

The same windowed statistic calculation was applied as the IMU change magnitude to compute the windowed distribution of F1 scores. FIG. 9A shows the distribution of F1 scores from different models in different sessions. Overall F1-score distributions from ATT-Conv1D and Contrast-Conv1D models are higher than Conv1D and ATT models. The ATT model's F1 score distribution shows improvement over the Conv1D model. The F1-score distributions between ATT-Conv1D and Contrast-Conv1D models are comparable, with one model performing better than the other in some sessions but not all. Across different sessions, F1-score distributions in sessions 4, 5, and 8 are among the lowest for the same model. This may be the result of high overall IMU change magnitudes, as illustrated in FIG. 8.

FIG. 9B shows the relationship between F1 scores and IMU change magnitudes from different models in the format of lineplot. The IMU change magnitudes are digitized into 100 bins for clarity purposes. F1-score distributions from all four models show a negative correlation with the IMU change magnitude, indicating ECG predictions become less accurate with more significant motion. The figures also illustrate a significant drop in F1 scores corresponding to extremely low IMU changes, which could correspond to sensors not picking up much information from dogs. Comparing figures for Conv1D, ATT, and ATT-Conv1D, they show a similar pattern that F1 scores drop drastically before IMU change magnitudes increase above 0.2, with ATT-Conv1D being most resilient to the performance decrease. The Contrast-Conv1D model shows a similar decrease in performance to the Conv1D model, but the decrease rate is lower.

Table 1 shows the overall performance measures of all four models across all sessions. Comparing raw signals, the ATT-Conv1D model produces the lowest MSE at 0.6637. Using the strict peak alignment parameters with 0.05 times sampling rate as the distance threshold and one error window tolerance, the ATT-Conv1D model also achieves the best performance. However, it is worth pointing out that the Contrast-Conv1D model's performance can also be considered comparable with the ATT-Conv1D model. If the distance threshold and four error window tolerance are used, the Contrast-Conv1D model's F1 score can be higher than the ATT-Conv1D models, as indicated by relax-F1 in Table 1. This is because while the Contrast-Conv1D model is capable of detecting QRS complexes even in the high motion scenario, its output contains significant noise in the QRS complexes, as illustrated in both FIGS. 6 and 7.

TABLE 1
Average quantitative performance over 10-fold CV

| Model | MSE | precision | recall | F1 | Relax-F1 |
|---|---|---|---|---|---|
| Conv1D | 0.9155 | 0.4145 | 0.2248 | 0.2875 | 0.3570 |
| ATT | 0.9298 | 0.4774 | 0.3925 | 0.4283 | 0.5334 |
| ATT-ConV1D | 0.6637 | 0.7747 | 0.6707 | 0.7181 | 0.7862 |
| Contrast-Conv1D | 0.7649 | 0.6841 | 0.5604 | 0.6112 | 0.8145 |

The experimental results suggest that both the attention-based model and the contrastive representation learning model can assist in alleviating the effect of motion artifacts and other noises, though potentially in different ways. The attention mechanism identifies highly correlated signals from the temporal windowed input itself through the self-attention matrix and increases their importance in ECG prediction. Both qualitative and quantitative results show that convolutional components can further improve its capability. On the other hand, contrastive representation learning filters out motion related noises by pushing embeddings corresponding to similar ECG wave patterns closer to each other and embeddings for different ECG wave patterns further away from each other. Such an approach modifies the underlying embedding distribution in its vector space for more effective downstream ECG prediction. The inspection of predicted ECG signals and the relaxed F1 score from the contrastive representation learning indicates that such an approach can generate R-peaks close to real ECG R-peaks and produce reasonable QRS complexes. However, its predicted ECG signals and F1 scores with the strict peak alignment parameters illustrate the high noise level in its predicted QRS complexes.

The noisy QRS complexes in the predicted ECG signals from the contrastive representation learning may be the result of how such a learning technique is adapted to the regression problem. Because the model input is generated by sliding windows across temporal sequences, this can result in a continuous and dense space for generated ECG segments. As a result, there may be no clear breaking points within the space of ECG segments, and the similarity can be high for ECG segments with slight yet visible differences. Such a phenomenon may contribute to the noises in predicted QRS complexes by the contrastive representation learning model since the predicted embedding for adjacent timestamps could be similar, especially when the input IMU feature itself contains noises from motion and other sources.

Referring next to FIG. 10, shown is an example of a processing circuitry 1000 that can be used for ECG signal reconstruction from IMU signals, in accordance with various embodiments of the present disclosure. The processing circuitry 1000 can comprise one or more computing/processing device such as, e.g., a smartphone, tablet, computer, controller, microcontroller, microprocessor, etc. The processing circuitry 1000 can include at least one processor circuit, for example, having a processor 1003 and a memory 1006, both of which are coupled to a local interface 1009. To this end, each processing circuitry 1000 may comprise, for example, at least one server computer or like device, which can be utilized in a cloud-based environment. The local interface 1009 may comprise, for example, a data bus with an accompanying address/control bus or other bus structure as can be appreciated.

Combining Attention-Based Models with Contrastive Representation Learning

The Attention-based Model and the Contrastive Representation Learning can be combined to achieve improved performance. Following the architecture in the contrastive representation learning, the embedding model can be a modified version of the attention model with the same output size as before while the downstream model architecture remains the same. Initial experiments suggest that while the base version of the attention model can reduce the adopted contrastive learning loss, the learned embedding does not contribute to effective ECG reconstruction in the downstream model. By combining the modified attention embedding model with the Contrastive Representation Learning, the model achieved a 0.8149 F1 score under the most restricted peak alignment condition.

Several structural changes can be made on the base Multi-Head Self Attention Block to incorporate contrastive representation learning. The output activation function of each block can be set to ReLU with an additional Batch Normalization applied before the activation function. In addition, instead of splitting query, key, and value matrices into multiple heads, only query and key matrices can be split while the value matrix can be broadcast across multiple heads. In other words, the value matrix is duplicated for each computed attention matrix to have a different temporal scale with the same features. The number of heads to split for query and key matrices is increased from 5 to 20 to allow the discovery of more temporal patterns. The dropout layers and pointwise feedforward layers are removed from the modified attention embedding model.

Additional experiments also show that while the residual operation can improve the learning speed in reducing the contrastive loss and retaining the position encoding information, it can negatively impact the effectiveness of learned embedding in ECG reconstruction. Therefore, the residual operation can be removed from the modified attention embedding model. To maintain the position encoding information, a relative position encoding mechanism can be adopted. Instead of adding the position encoding to input features, it is included as an additional input feature and passed to attention mechanism blocks. In addition to the dot product between query and key matrices, the attention weight matrix is also added by the dot product between the query matrix and a processed position encoding matrix. The processed position encoding matrix is the result of passing the position encoding through a Dense layer.

A lower IMU sensor frequency may also be incorporated while retaining a close model performance. A lower sensor frequency reduces the hardware requirement of the final product. Current experiments of lowering IMU signal frequency through random down sampling suggest the Contrastive Representation Learning can retain a similar peak alignment performance with 80 Hz but Attention-based Model's performance decreases significantly.

### Embodiments

In some embodiments, the processing circuitry 1000 can include one or more network interfaces 1012. The network interface 1012 may comprise, for example, a wireless transmitter, a wireless transceiver, and/or a wireless receiver. The network interface 1012 can communicate to a remote computing/processing device or other components using a Bluetooth®, WiFi, cellular or other appropriate wireless protocol. As one skilled in the art can appreciate, other wireless protocols may be used in the various embodiments of the present disclosure. The network interface 1012 can also be configured for communications through wired connections.

Stored in the memory 1006 are both data and several components that are executable by the processor(s) 1003. In particular, stored in the memory 1006 and executable by the processor 1003 can be an ECG reconstruction application 1015 which can reconstruct ECG signal from IMU signals as disclosed herein, and potentially other applications 1018. In this respect, the term “executable” means a program file that is in a form that can ultimately be run by the processor(s) 1003. Also stored in the memory 1006 may be a data store 1021 and other data. In addition, an operating system may be stored in the memory 1006 and executable by the processor(s) 1003. It is understood that there may be other applications that are stored in the memory 1006 and are executable by the processor(s) 1003 as can be appreciated.

Examples of executable programs may be, for example, a compiled program that can be translated into machine code in a format that can be loaded into a random access portion of the memory 1006 and run by the processor(s) 1003, source code that may be expressed in proper format such as object code that is capable of being loaded into a random access portion of the memory 1006 and executed by the processor(s) 1003, or source code that may be interpreted by another executable program to generate instructions in a random access portion of the memory 1006 to be executed by the processor(s) 1003, etc. Where any component discussed herein is implemented in the form of software, any one of a number of programming languages may be employed such as, for example, C, C++, C#, Objective C, Java®, JavaScript®, Perl, PHP, Visual Basic®, Python®, Ruby, Flash®, or other programming languages.

The memory 1006 is defined herein as including both volatile and nonvolatile memory and data storage components. Volatile components are those that do not retain data values upon loss of power. Nonvolatile components are those that retain data upon a loss of power. Thus, the memory 1006 may comprise, for example, random access memory (RAM), read-only memory (ROM), hard disk drives, solid-state drives, USB flash drives, memory cards accessed via a memory card reader, floppy disks accessed via an associated floppy disk drive, optical discs accessed via an optical disc drive, magnetic tapes accessed via an appropriate tape drive, and/or other memory components, or a combination of any two or more of these memory components. In addition, the RAM may comprise, for example, static random access memory (SRAM), dynamic random access memory (DRAM), or magnetic random access memory (MRAM) and other such devices. The ROM may comprise, for example, a programmable read-only memory (PROM), an erasable programmable read-only memory (EPROM), an electrically erasable programmable read-only memory (EEPROM), or other like memory device.

Also, the processor 1003 may represent multiple processors 1003 and/or multiple processor cores, and the memory 1006 may represent multiple memories 1006 that operate in parallel processing circuits, respectively. In such a case, the local interface 1009 may be an appropriate network that facilitates communication between any two of the multiple processors 1003, between any processor 1003 and any of the memories 1006, or between any two of the memories 1006, etc. The local interface 1009 may comprise additional systems designed to coordinate this communication, including, for example, ultrasound or other devices. The processor 1003 may be of electrical or of some other available construction.

Although the ECG reconstruction application 1015, and other various applications 1018 described herein may be embodied in software or code executed by general purpose hardware as discussed above, as an alternative the same may also be embodied in dedicated hardware or a combination of software/general purpose hardware and dedicated hardware. If embodied in dedicated hardware, each can be implemented as a circuit or state machine that employs any one of or a combination of a number of technologies. These technologies may include, but are not limited to, discrete logic circuits having logic gates for implementing various logic functions upon an application of one or more data signals, application specific integrated circuits (ASICs) having appropriate logic gates, field-programmable gate arrays (FPGAs), or other components, etc. Such technologies are generally well known by those skilled in the art and, consequently, are not described in detail herein.

Also, any logic or application described herein, including the laser ablation control application 1015, that comprises software or code can be embodied in any non-transitory computer-readable medium for use by or in connection with an instruction execution system such as, for example, a processor 1003 in a computer system or other system. In this sense, the logic may comprise, for example, statements including instructions and declarations that can be fetched from the computer-readable medium and executed by the instruction execution system. In the context of the present disclosure, a “computer-readable medium” can be any medium that can contain, store, or maintain the logic or application described herein for use by or in connection with the instruction execution system.

The computer-readable medium can comprise any one of many physical media such as, for example, magnetic, optical, or semiconductor media. More specific examples of a suitable computer-readable medium would include, but are not limited to, magnetic tapes, magnetic floppy diskettes, magnetic hard drives, memory cards, solid-state drives, USB flash drives, or optical discs. Also, the computer-readable medium may be a random access memory (RAM) including, for example, static random access memory (SRAM) and dynamic random access memory (DRAM), or magnetic random access memory (MRAM). In addition, the computer-readable medium may be a read-only memory (ROM), a programmable read-only memory (PROM), an erasable programmable read-only memory (EPROM), an electrically erasable programmable read-only memory (EEPROM), or other type of memory device.

Further, any logic or application described herein, including the ECG reconstruction application 1015, may be implemented and structured in a variety of ways. For example, one or more applications described may be implemented as modules or components of a single application. For example, the ECG reconstruction application 1015 can include a wide range of modules such as, e.g., an initial model or other modules that can provide specific functionality for the disclosed methodology. Further, one or more applications described herein may be executed in shared or separate computing/processing devices or a combination thereof. For example, a plurality of the applications described herein may execute in the same processing circuitry 1000, or in multiple computing/processing devices in the same computing environment. To this end, each processing circuitry 1000 may comprise, for example, at least one server computer or like device, which can be utilized in a cloud-based environment.

A machine learning framework to reconstruct ECG signals from inertial measurements obtained during dogs' temperament evaluation tests has been presented. Such a framework can provide vital information about the dog without adding additional burden to the dog and the extra effort of equipping the wearable device with ECG sensors. Models based on two machine learning techniques have been demonstrated and show improved performance on ECG reconstruction under heavy movements of the dogs compared to baseline models. The first model combines the attention mechanism with convolutional components, and it showed the best performance in the R-peak alignment with an F1 score of 0.7181. The second model utilizes a regression-based contrastive representation learning framework to learn an embedding for the downstream ECG reconstruction task. While it only achieved an 0.6112 F1 score, the qualitative and quantitative analysis suggested the second model's performance is on par with the first model but with a significant amount of noise in predicted QRS complexes of the ECG waveform.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

The term “substantially” is meant to permit deviations from the descriptive term that don't negatively impact the intended purpose. Descriptive terms are implicitly understood to be modified by the word substantially, even if the term is not explicitly modified by the word substantially.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. The term “about” can include traditional rounding according to significant figures of numerical values. In addition, the phrase “about ‘x’ to ‘y’” includes “about ‘x’ to about ‘y’”.

1. A method for reconstructing canine electrocardiogram (ECG) signals from Inertial Measurement Unit (IMU) data, comprising:
obtaining a plurality of seismocardiogram (SGD) signals from one or more IMU attached to a canine, the plurality of SGD signals comprising a first subset of signals generated by the one or more IMU prior to a target timestamp and a second subset of signals generated by the one or more IMU after the target timestamp;
extracting a defined set of input features from each of the plurality of SGD signals; and
generating reconstructed ECG signals from the defined set of extracted input features for each of the plurality of SGD signals using at least one neural network, each reconstructed ECG signal corresponding to a SGD signal of the plurality of SGD signals, wherein the reconstructed ECG signals represent an ECG reading of the canine.

2. The method of claim 1, further comprising rendering the reconstructed ECG signals for display on a device.

3. The method of claim 1, wherein the at least one neural network comprises an attention-based model.

4. The method of claim 3, wherein the attention-based model comprises a self-attention mechanism comprising feature extraction layers configured to extract query, value and key components.

5. The method of claim 4, wherein the attention-based model further comprises a point-wise feed forward layer configured to generate the reconstructed ECG signals based upon an output of the self-attention mechanism.

6. The method of claim 1, wherein the at least one neural network comprises a contrastive representation model.

7. The method of claim 6, wherein the contrastive representation model comprises an embedding model configured to determine contrastive loss between data pairs and a downstream model configured to generate the reconstructed ECG signals based upon an output of the embedding model.

8. The method of claim 1, wherein the plurality of SGD signals comprise three-axis accelerometer signals and three-axis gyroscope signals.

9. The method of claim 8, wherein the defined set of extracted input features comprises normalized three-axis accelerometer signals and normalized three-axis gyroscope signals.

10. A system for reconstructing canine electrocardiogram (ECG) signals from Inertial Measurement Unit (IMU) data, comprising:
one or more IMU configured for attachment to a canine, the one or more IMU configured to provide seismocardiogram (SGD) signals; and
processing circuitry configured to:
receive a plurality of SGD signals from the one or more IMU, the plurality of SGD signals comprising a first subset of signals generated by the one or more IMU prior to a target timestamp and a second subset of signals generated by the one or more IMU after the target timestamp;
extract a defined set of input features from each of the plurality of SGD signals; and
generate reconstructed ECG signals from the defined set of extracted input features for each of the plurality of SGD signals using at least one neural network, each reconstructed ECG signal corresponding to a SGD signal of the plurality of SGD signals, wherein the reconstructed ECG signals represent an ECG reading of the canine.

11. The system of claim 10, wherein the SGD signals are wirelessly transmitted to the processing circuitry.

12. The system of claim 11, comprising a harness configured to secure the one or more IMU to the canine, the harness comprising sensor circuitry configured to transmit the SGD signals.

13. The system of claim 10, wherein the plurality of SGD signals comprise three-axis accelerometer signals and three-axis gyroscope signals.

14. The system of claim 13, wherein the defined set of extracted input features comprises normalized three-axis accelerometer signals and normalized three-axis gyroscope signals.

15. The system of claim 10, wherein the at least one neural network comprises an attention-based model.

16. The system of claim 15, wherein the attention-based model comprises a self-attention mechanism comprising feature extraction layers configured to extract query, value and key components.

17. The system of claim 16, wherein the attention-based model further comprises a point-wise feed forward layer configured to generate the reconstructed ECG signals based upon an output of the self-attention mechanism.

18. The system of claim 10, wherein the at least one neural network comprises a contrastive representation model.

19. The system of claim 18, wherein the contrastive representation model comprises an embedding model configured to determine contrastive loss between data pairs and a downstream model configured to generate the reconstructed ECG signals based upon an output of the embedding model.

20. The system of claim 10, wherein the processing circuitry is further configured to render the reconstructed ECG signals for display on a device.
